(19) United States
(12) Patent Application Publication
(10) Pub. No.: US 2005/0142176 A1
(43) Pub. Date: Jun. 30, 2005

(54) Transdermal patch for long-term steady release

(75) Inventors: Ae-June Wang, Hsinchu City (TW); Li-Fen Yao, Hsinchu City (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsinchu (TW)

(21) Appl. No.: 11/023,625

(22) Filed: Dec. 29, 2004

(30) Foreign Application Priority Data
Dec. 31, 2003 (TW) 092137777

(51) Int. Cl. A61K 9/70; A61K 31/5513; A61K 31/445; A61K 31/21

(52) U.S. Cl. 424/449; 514/221; 514/304; 514/317; 514/509

(57) ABSTRACT

A patch containing at least one drug component is disclosed. The patch includes: a protecting membrane; a drug reservior layer containing a first concentration of the drug; an adhesion layer containing a second concentration of the drug and being in contact with the skin; and a release liner; wherein the drug reservior layer lying between the protecting membrane and the adhesion layer, and the first concentration being higher than the second concentration so as to steadily release the drug component by the diffusion caused by the difference between the first and second concentration.

### BACKGROUND OF THE INVENTION

1. Field of the Invention

The present invention relates to a transdermal patch and, more particularly, to a transdermal patch for long-term steady release.

2. Description of Related Art

Since ancient times in China, plaster has been pasted on the skin of a patient, with its drug component being absorbed and penetrated to skin for the therapy. Nowadays, the new transdermal patch—a high technology product also feature the same therory of those original Chinese plasters. The difference in the drug effect from the traditional plaster is the new transdermal formulation being absorbed steadily and delivered systemically to the whole body via the blood circulation to accomplish the therapeutic efficacy. The advantage is capability of avoiding first pass effect enhancing the bioavailability of the drug and reducing the side effect. Furthermore, for the chronic disease patients, it can also avoid the pain and the inconvenience experienced with long-term intravenous injections.

In another aspect, the transdermal formulation is steady release in the therapy due to its stable permeation rate to the skin for the maintenance of the drug effective concentration in the blood. Should the patients feel uncomfortable, the therapeutic process can be discontinued immediately without the interference of the remaining drug in the body. Therefore, the convenience in usage increases the patient cooperation factor. Moreover, the disorder of illness recurrence caused by lapses in taking medication, such as through forgetfulness, can be avoided.

Presently, there are three types of well-developed transdermal patches in the market including using one patch daily, two patches weekly, and one patch weekly. For the chronic and long-term therapeutic patients, the above transdermal patches could be a good choice. In terms of the construction patch types, the matrix type patch is the mainstream choice nowadays. In addition to its small size and the more comfortable usage to the patients, the matrix type patch further reduces the occurrence of skin allergy. Hypertension therapy, for example, generally is administrated orally every day, but the patients often forget to take the medication. In addition, there is another drawback of the oral delivery in that the medication may result in gastrointestinal tract irritation. Therefore, to develop the one week patch for anti-hypertension therapy, such as clonidine patch will not only provide a more convenient administration route for patients, but also reduce the side effect of the drug.

Currently, the only anti-hypertension patch on the market is manufactured by Boehringer Ingelheim Company, and the skill is initially developed from Alza Company. In this skill, a porous membrane is used to control the drug released rate. However, the use of the porous rate control membrane will increase the cost and complicate the preparing process compared to other art, and thus is unfavorable for commercialization.

### SUMMARY OF THE INVENTION

The object of the present invention is to provide a transdermal patch for long-term steady drug release.

Further objects are to shorten the production time, and to simplify the manufacturing process to reduce the cost of transdermal patch and maintain the same administration effect for commercialization.

A yet further object of the present invention is to provide a transdermal patch for replacing the use of a porous rate control membrane, and reaching the rate control effect simultaneously. To achieve the object, the present transdermal patch contains at least one drug component. The patch comprises: a protecting membrane, a drug reservoir layer containing a first concentration of the drug, an adhesion layer containing a second concentration of the drug and being in contact with the skin, and a release liner layer. Wherein, the drug reservoir layer lies between the protecting membrane and the adhesion layer, and the first concentration is higher than the second concentration so as to steadily release the drug component by the diffusion caused by the difference between the first and second concentrations.

Wherein the drug concentration of the first concentration is preferably higher than that of the second concentration. The preferred content of the first concentration drug component is of 5-12%-wt, and the preferred content of the second concentration drug component is of 1-4%-wt. The first excipient and the second excipient may be the same or not.

FIG. 1 is a sectional drawing of the present transdermal patch. Herein, the drug reservior layer 1 associates with the adhesion layer 2 mutually. As the above-mentioned, the drug concentration is different between the drug reservior layer 1 and the adhesion layer 2, that is, the drug concentration is high in the drug reservior layer 1, and the drug concentration is low in the adhesion layer 2. The purpose for the drug concentration design herein is due to the adhesion layer being in contact with the skin directly, and to avoid the disorders such as skin allergy, excessively-rapid drug release, and the first-pass effect caused by the high drug concentration, hence to lower the concentration. Further, the drug component of the high concentration drug reservior layer 1 can be steadily released in the gel state by the diffusion to the adhesion layer 2 caused by the gradient migration between the first and second concentrations, and then absorbed through the skin. In the figure, a release liner layer 4 and a protecting membrane 3 are further shown. The release liner layer 4 is peeled off before use, and the protecting membrane 3 is used to protect the drug reservior layer 1 from the damage of the ambient pressure and humidity.

Other objects, advantages, and novel features of the invention will become more apparent from the following detailed description when taken in conjunction with the accompanying drawings.

### BRIEF DESCRIPTION OF THE DRAWINGS

FIG. 1 is the sectional drawing of the transdermal patch in the present invention.

FIG. 2 is the variation of the drug release amount versus time of the transdermal patch from the embodiment 1 in the present invention.

FIG. 3 is the variation of the cumulative drug release amount versus time of the transdermal patch from the embodiment 1 in the present invention.

FIG. 4 is the variation of the drug release amount versus time of the transdermal patch from the embodiments 2, 3, and 4 in the present invention.

FIG. 5 is the variation of the cumulative drug release amount versus time of the transdermal patch from the embodiments 2, 3, and 4 in the present invention.

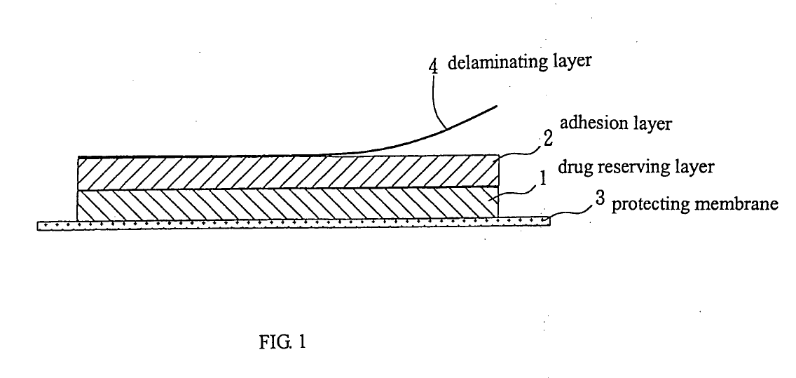

### DETAILED DESCRIPTION OF THE PREFERRED EMBODIMENT

In the preferred embodiment of the present invention, the drug reservior layer contains the first concentration drug component, and the suitable first excipient is further added. Wherein the drug component could be any one suitable for transdermal administration, such as clonidine, fentanyl, scopolamine, naloxone, ketamine, benzodiazepines, oxybutynin, lesopitron, estradiol, levonorgestrel, albuterol, labetolol, atropine, haloperidol, isosorbide dinitrate, nitroglycerin, norethindrone acetate, nicotine, benztropine, secoverine, dexsecoverine, and arecoline. The excipient is chosen appropriately with its component comprising: drug component carrier, surfactant, filler, and polymer matrix. The suitable drug component carrier is selected from a common group comprising: light mineral oil, myristates, isostearates, glycerides, polyethylene glycol and the derivative thereof, and the mixture thereof. The suitable surfactant comprises: vitamin E and the derivative thereof, oleic acid and the derivative thereof, and the mixture thereof. The suitable filler is a commonly available one, such as silicone dioxide. The polymer matrix serves as the adhesive, hence the common use comprises: Acry series adhesive polymer and polyisobutylene polymer. The present adhesion layer with its component contains the second concentration drug component and the suitable second excipient, wherein the drug component could be any one suitable for transdermal administration, such as clonidine, fentanyl, scopolamine, naloxone, ketamine, benzodiazepines, oxybutynin, lesopitron, estradiol, levonorgestrel, albuterol, labetolol, atropine, haloperidol, isosorbide dinitrate, nitroglycerin, norethindrone acetate, nicotine, benztropine, secoverine, dexsecoverine, and arecoline. The excipient is chosen appropriately with its component comprising: a drug component carrier, surfactant, filler, and polymer matrix. The suitable drug component carrier is selected from a general group comprising: light mineral oil, myristates, isostearates, glycerides, polyethylene glycol and the derivative thereof, and the mixture thereof. The suitable surfactant comprises: vitamin E and the derivative thereof, oleic acid and the derivative thereof, and the mixture thereof. The suitable filler is a commonly available one, such as silicone dioxide. The polymer matrix serves as the adhesive, hence the common use comprises: Acry series adhesive polymer and polyisobutylene polymer.

For the better understanding of the skilled art in the present invention, there are five preferred specific embodiments described as follows. In the following embodiments, the drug component of the first concentration and the second concentration is exemplified by the anti-hypertension drug, namely clonidine.

### Embodiment 1

Step 1 Gel Preparation

A 9%-wt of Oppanol B-100 (BASF Company; polyisobutenes with molar mass of 250,000) and a 12%-wt of Oppanol B-10 (BASF Company; polyisobutenes with molar mass of 24,000) are placed in a 5L stirring tank. Cyclohexane is added as a solvent and stir for one day, and then the mixture is moved to the rolling mixer for rolling another one day to produce a transparent gel, namely Oppanol B gel.

Step 2 Mixing of the R Layer/A Layer Formulation

The formulation comprises the R layer (drug reservior layer) mixing process and the A layer (adhesion layer) mixing process.

1. R Layer Mixing Process:

A 0.5%-wt of silicone dioxide is added to a 39%-wt of light mineral oil, and then the mixture is shaken with Vortex until an emulsion is presented. Then, a 110%-wt of clonidine material is added to the mixture for further shaking with Vortex. After an emulsion is presented, the mixture is mixed for 24 hrs in the rolling mixer. The 50.5%-wt of Oppanol B gel from embodiment 1 is added to the mixture, and then mixed with the rolling mixer for another 24 hrs to produce a milky-white colored gel, namely R layer mixture.

2. A Layer Mixing Process:

A 0.5%-wt of silicone dioxide is added to a 54%-wt of light mineral oil, and then the mixture is shaken with Vortex until an emulsion is presented. Then, a 2%-wt of clonidine material is added to the mixture for another shaking with Vortex. After an emulsion is presented, the mixture is mixed for 24 hrs in the rolling mixer. The 43.5%-wt of Oppanol B gel is added to the mixture, and then mixed with the rolling mixer for another 24 hrs to produce a milky-white colored gel, namely A layer mixture.

Step 3 Coating, Drying and Laminating (C/D/L)

The coating, drying and laminating comprise the R layer coating process and the R/A layer coating process.

1. R Layer Coating Process:

A protecting membrane is installed in the material axle of the coating/drying/laminating machine, and a 3M release liner layer is installed in the laminating axle. Then, the machine is turned on, and the condition is set on the tension controller panel. Further, the coating temperature of the coating/drying/laminating machine is set. The coating comprises two stages in temperature control, including a first stage of 60-80° C., and a second stage of 80-110° C. The R layer coating process starts once the set temperature is reached. The R layer mixture produced from the mixing process is poured into the coating tank for coating, drying and laminating. After passing through the drying cabinet from the coating tank to the laminating section, the R layer gel is laminated with the release liner layer and then rolled up on the roller to produce a gel roll, namely R layer drug roll.

2. R/A Layer Coating Process:

A release liner layer is installed in the material axle of the coating dry laminator, and the R layer drug roll from the R layer coating is installed in the laminating axle. Then, the laminator is actuated, and the condition is set on the tension controller panel. Further, the coating temperature of the coating dry laminator is set. The coating comprises two stages in temperature control, including a first stage of 60-80° C., and a second stage of 80-110° C. The RIA layer coating process starts once the set temperature is reached. The A layer mixture produced from the mixing process is poured into the coating tank for coating, drying and laminating. After passing through the drying cabinet from the coating tank to the laminating section, the R layer drug roll (the release liner layer is peeled off and gathered by the scrap axle) is laminated with the A layer gel and then rolled up on the roller to produce a gel roll, namely R/A layer drug roll.

Step 4 Slitting

The slitting comprises the release liner layer slitting process and the R/A layer drug roll slitting process.

1. Release Liner Layer Slitting Process:

A slitter is used for the release liner layer slitting process with the slitting width set as 2.5 cm into 4 strips. Then, the slitter is actuated for slitting and rolling up the produced release liner layer on the roller.

2. R/A Layer Drug Roll Slitting Process:

A slitter is used for the R/A layer drug roll slitting process with the slitting width set as 2.5 cm into 4 strips. Then, the slitter is actuated for slitting and rolling up the produced R/A layer drug roll on the roller.

Step 5 Patch Slicing and Packaging

The slit drug roll is installed in the material axle, and then copperplate aluminum foil packaging paper is installed in the packaging material axle. Further, the temperature, tension, and RUN CMD2 conditions of the patch-slicing packer are set. The power, temperature control, material, and scrap are set. The patch slicing and packaging process are started once the set temperature is reached. In the process, the formed release liner scrap is rolled up on the scrap axle to produce the end products.

### Embodiments 2 to 4

Based on steps 1 to 5 of the embodiment 1 in the present invention, embodiments 2 to 4 change the R layer (the drug reservior layer) and the A layer (the adhesion layer) formulation in the step 2 mixing process and are described as weight percentage in the following Table 1.

TABLE 1
The formulation of embodiments 2 to 4

| Formulation with additive\%-wt | | | Embodiment 2 | Embodiment 3 | Embodiment 4 |
|---|---|---|---|---|---|
| R layer | Drug | Clonidine | 9.4 | 9.4 | 9.4 |
| | component | | | | |
| | Excipient | Light Mineral | 40.3 | 37.3 | 35.3 |
| | | Oil | | | |
| | | Oppanol B gel | 50.3 | 50.3 | 50.3 |
| | | SiO₂ | — | 3 | 5 |
| A layer | Drug | Clonidine | 2.8 | 2.8 | 2.8 |
| | component | | | | |
| | Excipient | Light Mineral | 54.1 | 51.1 | 49.1 |
| | | Oil | | | |
| | | Oppanol B gel | 43.1 | 43.1 | 43.1 |
| | | SiO₂ | — | 3 | 5 |

### Embodiments 5 to 10

Based on steps 1 to 5 of embodiment 1 in the present invention, embodiments 5 to 10 change the R layer (the drug reservior layer) and the A layer (the adhesion layer) formulation in the step 2 mixing process and are described as weight percentage in the following Table 2.

TABLE 2
The formulation of embodiments 5 to 10

| Formulation with additive\ | | | | | | | | Embodiment |
|---|---|---|---|---|---|---|---|---|
| %-wt | | | Embodiment 5 | Embodiment 6 | Embodiment 7 | Embodiment 8 | Embodiment 9 | 10 |
| R | Drug | Clonidine | 9.0 | 9.0 | 9.0 | 9.0 | 9.0 | 9.0 |
| layer | component | | | | | | | |
| | Excipient | Light | 25.8 | 37.8 | 37.8 | — | — | — |
| | | Mineral | | | | | | |
| | | Oil | | | | | | |
| | | 10% | 13.0 | — | — | — | — | — |
| | | TPGS | | | | | | |
| | | Coster | — | — | — | 38.8 | — | — |
| | | 5024 | | | | | | |
| | | Coster | — | — | — | — | 38.8 | — |
| | | 5088 | | | | | | |
| | | Larrafil | — | — | — | — | — | 38.8 |
| | | M 1944 | | | | | | |
| | | CS | | | | | | |
| | | Oppanol | 52.2 | 52.2 | 52.2 | 52.2 | 52.2 | 52.2 |
| | | B gel | | | | | | |
| | | 1% Span | — | 1.0 | — | — | — | — |
| | | 80 | | | | | | |
| | | 1% PEG | | | 1.0 | — | — | — |
| | | 400 | | | | | | |
| | | Clonidine | 9.0 | 9.0 | 9.0 | 9.0 | 9.0 | 9.0 |
| A | Drug | Clonidine | 2.7 | 2.7 | 2.7 | 2.7 | 2.7 | 2.7 |
| layer | component | | | | | | | |
| | Excipient | Light | 34.9 | 51.4 | 51.4 | — | — | — |
| | | Mineral | | | | | | |
| | | Oil | | | | | | |
| | | 10% | 17.5 | — | — | — | — | — |
| | | TPGS | | | | | | |
| | | Coster | — | — | — | 52.4 | — | — |
| | | 5024 | | | | | | |
| | | Coster | — | — | — | — | 52.4 | — |
| | | 5088 | | | | | | |
| | | Larrafil | — | — | — | — | — | 52.4 |
| | | M 1944 | | | | | | |
| | | CS | | | | | | |
| | | Oppanol | 44.9 | 44.9 | 44.9 | 44.9 | 44.9 | 44.9 |
| | | B gel | | | | | | |
| | | 1% Span | — | 1.0 | — | — | — | — |
| | | 80 | | | | | | |
| | | 1% PEG | — | — | 1.0 | — | — | — |
| | | 400 | | | | | | |
| | | Clonidine | 2.7 | 2.7 | 2.7 | 2.7 | 2.7 | 2.7 |

Wherein:
TPGS: vitamin E derivative (alpha-tocopherol polyethylene glycol succinate).
Coster 5024: a merchandise of 2-octyldodecyl myristate.
Coster 5088: a merchandise of isostearyl isostearate.
Larrafil M 1944 CS: a merchandise of oleoyl macrogol-6 glycerides.
Span 80: a merchandise of sorbitan monooleate.
PEG 400: a merchandise of polyethylene glycol (MW: 380-420)

### Embodiment 11

### In vitro Skin Penetration Test

For the effectiveness of the present transdermal patch, the in vitro skin permeation test is performed as follows.

Process of the In Vitro Skin Permeation Test

1. Materials and Agents:

Derma: human skin.

Dermal penetration device: transparent permeation device (Modified Franz Diffusion Cell).

2. Methods:

(1) Add pH 7.4 PBS (phosphate buffer saline) extraction buffer and the stir bar together in the transparent permeation device (Modified Franz Diffusion Cell), and then put them in the heating device under the set temperature of 32±0.5° C. with stirring.

(2) Take the prepared skin, and unfreeze it at room temperature. Then, assemble the skin permeation device, and fix the skin sample with an iron clamp.

(3) Begin to record the time, and collect the samples at the particular time point.

(4) The collected sample is analyzed by HPLC, and the drug concentration is calculated according to the established calibration curve. Hence, the flux per hour per unit area, and the cumulative amount per unit area can be calculated.

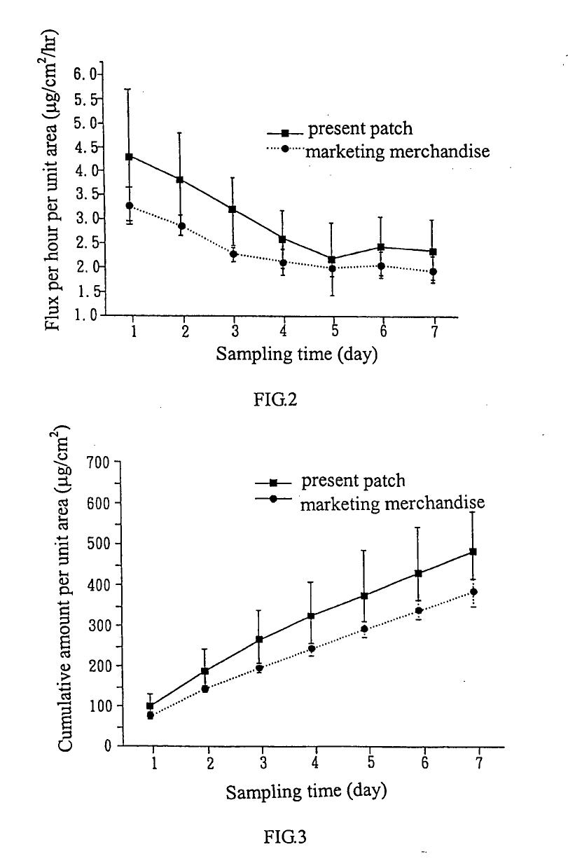

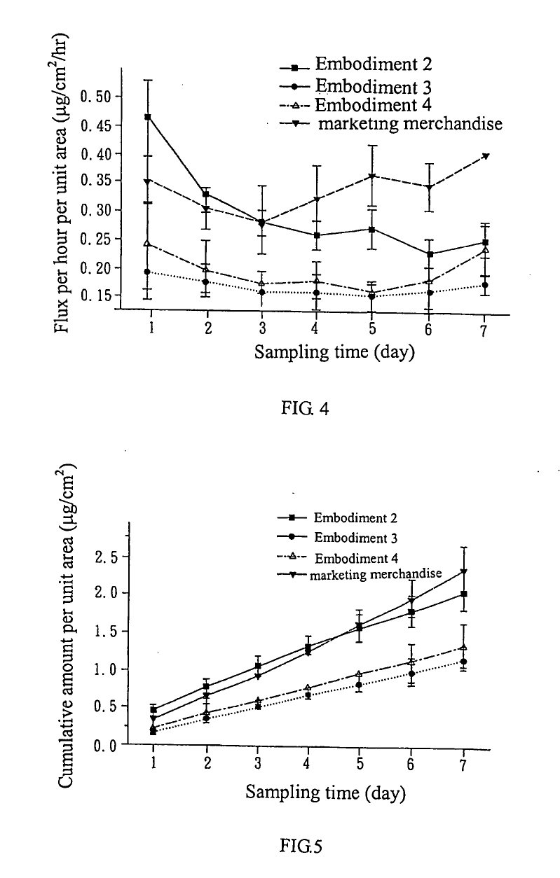

The patch produced from the step 1 to 5 of the embodiment 1 formulation is compared to the merchandise of Boehringer Ingelheim Company, in the flux per hour per unit area (μg/cm²/hr) and the cumulative amount per unit area (μg/cm²) of the 7 days with the in vitro skin permeation test. It is suggested that the drug release concentration in the blood is comparable to the commercialized anti-hypertension patch. The results are shown in Table 3 and FIG. 2.

Table 3 is the comparison between the present in vitro study and the merchandise in the market, including the mean drug release amount per day and per hour. The result shows that the mean drug release amount per hour of the present embodiment 1 patch is better than that of the marketing merchandise.

TABLE 3
The flux per hour per unit area
Flux per hour per unit area (μg/cm²/hr)

| | Embodiment 1 patch | Commercialized patch |
|---|---|---|
| Day | μg/cm²/hr | μg/cm²/hr |
| 1 | 4.27 | 3.26 |
| 2 | 3.80 | 2.84 |
| 3 | 3.17 | 2.24 |
| 4 | 2.55 | 2.08 |
| 5 | 2.13 | 1.97 |
| 6 | 2.38 | 1.99 |
| 7 | 2.30 | 1.90 |

The results of the cumulative drug release amount are shown in Table 4 and FIG. 3. It is suggested that the patch of the present embodiment 1 provides a steady drug release, and the cumulative drug release amount is even better than that of the marketing merchandise.

TABLE 4
The cumulative amount per unit area (μg/cm²)
Cumulative amount per unit area (μg/cm²)

| | Embodiment 1 | |
|---|---|---|
| | patch | Commercialized patch |
| Day | μg/cm² | μg/cm² |
| 1 | 102.52 | 78.12 |
| 2 | 193.62 | 146.38 |
| 3 | 269.59 | 200.15 |
| 4 | 330.85 | 250.03 |
| 5 | 382.03 | 297.37 |
| 6 | 439.06 | 345.12 |
| 7 | 494.33 | 390.81 |

Similarly, the patches of embodiments 2, 3 and 4 based on steps 1 to of the embodiment 1 are compared to the merchandise of Boehringer Ingelheim Company, in the flux per hour per unit area (μg/cm²/hr) and the cumulative amount per unit area (μg/cm²) of the 7 days from the in vitro skin permeation test. It is suggested that the drug release concentration of the invention is higher than that of the commercialized anti-hypertension patch. The result is shown in Table 4. The cumulative amount per unit area (μg/cm²) is the comparison of the cumulative drug release amount between the present in vitro study and the marketing merchandise. The result in Table 5 shows that compared to the merchandise in the market, the present patch provides a better cumulative drug release amount than that of the marketing merchandise.

### Embodiment 12

### Toxicity of the Administration

The embodiment aims to study whether the toxicity from the present transdermal patch could be accepted for a human body.

1. Animal Dermal Sensitization and Skin Irritation Test

The patch from the embodiment 1 is commissioned to the Northview Pacific Laboratories, Inc. USA for the animal dermal sensitization test and the skin irritation test. Those tests are described as follows:

(1) Dermal Sensitization Test

The test follows the Northview standard operating procedure 16G-12. Using the Buehler method, 48 guinea pigs 6 weeks old with 300-500 g in body weight are tested for 7 days to observe whether the test drug could induce the red and swollen skin. The result shows that the present patch has no potential dermal sensitization in the test animals.

(2) Skin Irritation Test

The test follows the Northview standard operating procedure 16G-44. Three female rabbits with 2.8-3.2 kg in body weight are tested for 7 days. After removing the test patch from the rabbits, the animals were observed for signs of red and swollen phenomenon in 1, 24, 48 and 72 hr, respectively. The result shows that the present patch has no skin irritation.

From the above-mentioned embodiments, it is realized that the transdermal patch in the present invention linearly releases the drug component to the blood, and even steadily maintains the effective drug concentration on the seventh day. It is evidenced that the double layer of the present invention can be used in the long-term steady drug release, and save the known rate control membrane. For the drug release rate or skin irritation/sensitization, the transdermal patch of the present invention is comparable to that of the known merchandize.

Although the present invention has been explained in relation to its preferred embodiment, it is to be understood that many other possible modifications and variations can be made without departing from the spirit and scope of the invention as hereinafter claimed.

1. A transdermal patch containing at least one drug component, comprising:
a protecting membrane;
a drug reservior layer, containing a first concentration of the drug;
an adhesion layer, containing a second concentration of the drug and being in contact with the skin; and
a release liner
wherein,
said drug reservior layer lying between said protecting membrane and said adhesion layer;
said adhesion layer lying between said drug reservior layer and said release liner; and
said first concentration being higher than said second concentration so as to steadily release said drug component by the diffusion caused by the difference between said first concentration and said second concentration.

2. The transdermal patch as claimed in claim 1, wherein at least one of said drug component is selected from the group comprising: clonidine, fentanyl, scopolamine, naloxone, ketamine, benzodiazepines, oxybutynin, lesopitron, estradiol, levonorgestrel, albuterol, labetolol, atropine, haloperidol, isosorbide dinitrate, nitroglycerin, norethindrone acetate, nicotine, benztropine, secoverine, dexsecoverine, and arecoline.

3. The transdermal patch as claimed in claim 2, wherein said drug reservior layer further comprises a first polymer matrix.

4. The transdermal patch as claimed in claim 3, wherein said drug reservior layer selectively comprises a first drug component carrier selected from a group consisting of light mineral oil, myristates, isostearates, glycerides, polyethylene glycol, and the derivative thereof, and a content of said first drug component carrier is 24 to 55%-wt of said drug reservior layer.

5. The transdermal patch as claimed in claim 3, wherein said drug reservior layer further comprises a first filler silicone dioxide with a content of at least 0.5%-wt of said drug reservior layer.

6. The transdermal patch as claimed in claim 3, wherein said first polymer matrix of said drug reservior layer is selected from a group consisting of Acry series adhesive polymer and polyisobutylene polymer, with a content of 15 to 80%-wt of said drug reservior layer.

7. The transdermal patch as claimed in claim 3, wherein said drug component of said drug reservior layer is clonidine having a content of 9 to 12%-wt of said drug reservior layer.

8. The transdermal patch as claimed in claim 7, wherein a first surfactant is selectively added to said drug reservior layer and said adhesion layer, and said first surfactant is selected from a group consisting of vitamin E and the derivative thereof, oleic acid and the derivative thereof, and the mixture thereof.

9. The transdermal patch as claimed in claim 2, wherein said adhesion layer further comprises a second polymer matrix.

10. The transdermal patch as claimed in claim 9, wherein said adhesion layer further selectively comprises a second drug component carrier selected from a group consisting of light mineral oil, myristates, isostearates, glycerides, polyethylene glycol and the derivative thereof, and the mixture thereof, and a content of said second drug component carrier is 40 to 71%-wt of said adhesion layer.

11. The transdermal patch as claimed in claim 9, wherein said second polymer matrix of said adhesion layer is selected from a group consisting of Acry series adhesive polymer and polyisobutylene polymer, with a content of 15 to 80%-wt of said drug reservior layer.

12. The transdermal patch as claimed in claim 9, wherein said adhesion layer further comprises a second excipient being silicone dioxide with a content of at least 0.5%-wt of said drug reservior layer.

13. The transdermal patch as claimed in claim 9, wherein said drug component of said adhesion layer is clonidine having a content of 9 to 12%-wt of said drug reservior layer.

14. The transdermal patch as claimed in claim 13, wherein a second surfactant is selectively added, and selected from a group consisting of vitamin E and the derivative thereof, oleic acid and the derivative thereof, and the mixture thereof.

15. A process for preparing a transdermal patch, said transdermal patch contains at least one drug component, comprising: a protecting membrane; a drug reservior layer containing a first concentration of the drug; an adhesion layer containing a second concentration of the drug and being in contact with the skin; and a release liner; wherein the drug reservior layer lying between the protecting membrane and the adhesion layer, and the first concentration being higher than the second concentration; and said process comprises:
(1) mixing a first polymer matrix and said drug component-contained first concentration to produce a drug reservior layer mixture;
(2) mixing a second polymer matrix and said drug component-contained second concentration to produce an adhesion layer mixture;
(3) providing a coating dry laminator, and coating said drug reservior layer mixture on a first release liner to produce a drug reservior layer; wherein said coating temperature is between 60 to 100° C. in the process; and
(4) coating said adhesion layer mixture on a second release liner to produce an adhesion layer, and laminating said adhesion layer on said drug reservior layer to produce a drug reservior layer/adhesion layer; wherein said coating temperature is between 60 to 100° C. in the process.

16. The preparing process as claimed in claim 15, wherein said drug reservior layer mixture further comprises a first drug component carrier with the content of 24 to 55%-wt; a first filler with a content of 0.5 to 2%-wt; and a first polymer matrix with a content of 15 to 80%-wt;
wherein,
said first concentration is of 9 to 12%-wt; said first drug component carrier is selected from a group consisting of light mineral oil, myristates, isostearates, glycerides, polyethylene glycol and the derivative thereof, and the mixture thereof; said first filler is silicone dioxide; said first polymer matrix is selected from a group consisting of Acry series adhesive polymer, polyisobutylene polymer, and the mixture thereof; and said drug component is clonidine.

17. The preparing process as claimed in claim 16, wherein a first surfactant is selectively added to step (1) and step (2), and said first surfactant is selected from a group consisting of vitamin E and the derivative thereof, oleic acid and the derivative thereof, and the mixture thereof.

18. The preparing process as claimed in claim 15, wherein said adhesion layer mixture further comprises a second drug component carrier with a content of 40 to 71%-wt; a second filler with a content of 0.5 to 2%-wt; and a second polymer matrix with a content of 15 to 80%-wt;
wherein,
said second concentration is of 1 to 4%-wt, and said second drug component carrier is selected from a group consisting of light mineral oil, myristates, isostearates, glycerides, polyethylene glycol and the derivative thereof, and the mixture thereof; said second filler is silicone dioxide; said second polymer matrix is selected from a group consisting of Acry series adhesive polymer, polyisobutylene polymer, and the mixture thereof; and said drug component is clonidine.

19. The preparing process as claimed in claim 18, wherein a second surfactant is selectively added to step (1) and step (2), and said second surfactant is selected from a group consisting of vitamin E and the derivative thereof, oleic acid and the derivative thereof, and the mixture thereof.
